(19) United States
(12) Patent Application Publication
(10) Pub. No.: US 2024/0020919 A1
(43) Pub. Date: Jan. 18, 2024

(54) METHOD AND APPARATUS FOR SUPPLYING A THREE-DIMENSIONAL MODEL OF AN OBJECT

(71) Applicant: Siemens Healthcare GmbH, Erlangen (DE)

(72) Inventor: Sebastian KRUEGER, Erlangen (DE)

(73) Assignee: Siemens Healthcare GmbH, Erlangen (DE)

(21) Appl. No.: 18/332,921

(22) Filed: Jun. 12, 2023

(30) Foreign Application Priority Data
Jun. 15, 2022 (EP) 22179088.4

(51) Int. Cl. G06T 17/00; G06T 7/12; H04N 13/275; G16H 30/40; G16H 50/50

(52) U.S. Cl. CPC G06T 17/00; G06T 7/12; H04N 13/275; G16H 30/40; G16H 50/50

(57) ABSTRACT

A method for supplying a three-dimensional model of an object represented by volume data, comprises: supplying the volume data; supplying an image synthesis algorithm, the image synthesis algorithm being configured for visualizing the three-dimensional object by mapping the volume data onto visualization pixels of a two-dimensional visualization image; ascertaining a parameter set for actuating the image synthesis algorithm, the parameter set being suitable for generating, with the image synthesis algorithm, a set of a plurality of different visualization images based on the volume data set; generating the set of the plurality of different visualization images by mapping the volume data, with the image synthesis algorithm, using the parameter set; calculating the 3D model based on the set of the plurality of different visualization images; and supplying the 3D model.

### CROSS-REFERENCE TO RELATED APPLICATION(S)

The present application claims priority under 35 U.S.C. § 119 to European Patent Application No. 22179088.4, filed Jun. 15, 2022, the entire contents of which are incorporated herein by reference.

### FIELD

One or more embodiments of the present invention relate to methods and apparatuses for generating and supplying a virtual three-dimensional model (3D model) of a real three-dimensional object. The 3D model is supplied for transfer to and use by a user who may have different views of the object displayed using the 3D model.

### BACKGROUND

The modeling, reconstruction or visualizing of three-dimensional objects has a wide area of application in the fields of medicine (for example CT, PET), physics (for example electron structure of large molecules) or geophysics (composition and position of the layers of the earth). Typically the object to be examined is irradiated (for example via electromagnetic waves or sound waves) to examine its composition. The scattered radiation is detected and properties of the object are ascertained from the detected values. The result conventionally consists of a physical variable (for example density, tissue type, elasticity, speed) whose value is ascertained for the object. As a rule, a virtual grid is used at whose grid points the value of the variable is ascertained. These grid points are conventionally referred to as voxels. The term “voxel” is a portmanteau formed from the terms “volume” and “pixel”. A voxel corresponds to the spatial coordinate of a grid point with which the value of a variable at this location is associated. This is usually a physical variable, which can be represented as a scalar or vectorial field, in other words, the corresponding field value is assigned to the spatial coordinate. The data of the object obtained in this way is also referred to as volume data. The value of the variable or of the field at any object points (in other words, at any locations of the examined object) can be obtained by interpolation of the voxels.

For visualizing the volume data, a three-dimensional representation of the object or body on a two-dimensional display area (for example a screen or a panel or lens of what are known as “augmented reality glasses”) is conventionally generated from the voxels. In other words, voxels (defined in three dimensions) are mapped onto pixels (defined in two dimensions) of a two-dimensional visualization image. The pixels of the visualization image will also be called visualization pixels below. The mapping is conventionally referred to as volume rendering. How information contained in the voxels is reproduced by the pixels depends on the implementation of the volume rendering.

One of the most-used methods for volume rendering is what is known as ray casting (cf. Levoy: “Display of Surfaces from Volume Data”, IEEE computer Graphics and Applications, Issue 8, No. 3, May 1988, pages 29-37). With ray casting, simulated rays, which emanate from the eye of an imaginary viewer, are sent through the examined body or the examined object. Along the rays RGBA values are determined from the voxels for sampling points and are combined via Alpha Compositing or Alpha Blending to form pixels for a two-dimensional image. In the expression RGBA the letters R, G and B stand for the color portions red, green and blue from which the color contribution of the corresponding sampling point is composed. A stands for the ALPHA value, which constitutes a measure of the transparency at the sampling point. The respective transparency is used in the overlaying of RGB values on sampling points relating to the pixel. Lighting effects are conventionally taken into account via a lighting model in the context of a method referred to as “shading”.

A further method for volume rendering is what is known as path tracing (cf. Kajiya: “The rendering equation”, ACM SIGGRAPH computer Graphics, Issue 20, No. 4, August 1986, pages 143-150). In this case, a plurality of simulated rays per visualization pixel is shot into the volume data, which then interact with the volume, in other words, are reflected, refracted or absorbed, with at least one random ray being generated each time (apart from in the case of absorption). Each simulated ray thus seeks its path through the volume data. The more virtual rays are used per visualization pixel, the more the ideal image is approximated. In particular, the methods and processes described in EP 3 178 068 B1 are applied. The content of EP 3 178 068 B1 is incorporated herein by reference in its entirety.

### SUMMARY

A primary technical obstacle in the implementation of a system for interactive volume rendering is the fast, efficient and local processing of large volumes of data. It is precisely on devices having limited computing or graphics power that fast volume rendering is a challenge. Examples of such devices are mobile devices such as smartphones or tablets. These are usually equipped with current-saving processors (“System on a Chip”), which, compared to modern PCs, have comparatively little computing power. Furthermore, the portability of a volume rendering system is often limited since both the volume rendering algorithm and the volume data have to be ported for local use. This is often not possible owing to the volumes of data and for reasons of data security. In addition, the discovery of suitable parameters for generating a suitable visualization image is often not trivial and is difficult for the end user to overlook since the end user is typically not an expert in methods of volume rendering.

The consequence of this is that interactive volume rendering by the end user is frequently possible to only a limited extent or not at all. As a rule, the user is only supplied with individual visualization images. However, it is precisely in the medical field that it is important that a user, for instance a doctor, can freely change the views in the volume rendering. It is often only in this way that it is possible to understand the underlying facts.

It is an object of one or more embodiments of the present invention to provide improved methods and apparatuses in this regard, which guarantee, in particular, better portability and interactivity of volume rendering of a three-dimensional object for a user.

This and further objects are achieved with a method, an apparatus, a non-transitory computer program product and/or a non-transitory computer-readable storage medium respectively as claimed and disclosed herein. Advantageous developments are disclosed in the dependent claims and described herein.

The inventive solution to the object will be described below both in relation to the claimed apparatuses and in relation to the claimed method. Features, advantages or alternative embodiments mentioned in this connection should likewise be transferred to the other claimed subject matters, and vice versa. In other words, the concrete claims (which are directed, for example, toward an apparatus) can also be developed with the features, which are described or claimed in connection with a method. The corresponding functional features of the method are embodied by corresponding concrete modules in this case.

According to one aspect, a computer-implemented method for supplying a 3D model of a three-dimensional object represented by volume data is supplied. The method has a plurality of steps. One step is directed toward supplying the volume data. A further step is directed toward supplying an image synthesis algorithm, which image synthesis algorithm is embodied for visualizing the three-dimensional object by mapping the volume data onto visualization pixels of a two-dimensional visualization image. A further step is directed towards ascertaining a parameter set for actuating the image synthesis algorithm, which parameter set is suitable for generating, with the image synthesis algorithm, a set of a plurality of different visualization images on the basis of the volume data set. A further step is directed toward generating the set of the plurality of different visualization images by mapping the volume data, with the image synthesis algorithm, using the parameter set. A further step is directed toward calculating the 3D model on the basis of the set of the plurality of visualization images. A further step is directed toward supplying the 3D model (for a user).

The volume data can have a plurality of voxels. A voxel (“volume pixel” or three-dimensional pixel) is a volume element, which represents a value on a regular grid in the three-dimensional (3D) space. Voxels are analogous to pixels, which represent two-dimensional (2D) image data. As with pixels the voxels themselves typically do not contain their position in the space (their coordinates), and instead their coordinates are derived on the basis of their positions relative to other voxels (i.e. their positions in the data structure, which forms a single volume image). The value of a voxel can represent different physical properties of the three-dimensional object, such as a local density. In CT scans the values are expressed, for example, in Hounsfield units, which represent the opacity of a mapped material in relation to X-rays. The volume data thereby describes a three-dimensional object in an object volume. In particular, the volume data can disclose a (in particular inhomogeneous) density of the three-dimensional object in the object volume.

The volume data can be supplied, in particular, by a medical imaging method. The imaging method can be based, for example, on radiography, computed tomography (CT), magnetic resonance tomography (MR), ultrasound and/or positron emission tomography (PET). The three-dimensional object can accordingly be a body or body part of a patient. The three-dimensional object can comprise one or more organ(s) of the patient.

The image synthesis algorithm can be conceived, in particular, as a computer program product, which is embodied for mapping the volume data onto a two-dimensional projection surface or for volume rendering of the three-dimensional object or for volume rendering of the three-dimensional object. The projection surface is given by the visualization image. The image synthesis algorithm can have program components in the form of one or more instruction(s) for a processor for calculating the visualization image. Other terms for image synthesis algorithm are, for example, “renderer”, “render algorithm” or “volume renderer”. The image synthesis algorithm can be supplied, for example, in that it is held available in a storage facility (also referred to a storage device or memory) or is loaded into a working memory of a suitable data processing facility (also referred to as a processing device) or is made available generally for use.

The image synthesis algorithm can implement different methods for visualizing a volume data set individually or in combination. For example, the image synthesis algorithm can have a ray casting module and/or a path tracing module. The image synthesis algorithm maps the volume data onto the visualization pixels.

Depending on the selected settings (parameters) the image synthesis algorithm can implement a particular view, perspective, scene illumination, transfer function, transparency of one or more region(s) of the volume data, a section plane through the volume data, a region of interest within the volume data, and/or one or more item(s) of additional information for fading into the visualization image for a visualization image. A transfer function comprises representation parameters. They associate a particular color, transparency, contrast, lighting, sharpness and the like with each value in the three-dimensional volume. Generally speaking the representation parameters influence the type of representation of objects of the corresponding object type in the visualization image output to the user.

A visualization image is the two-dimensional result of the volume rendering process. The visualization image is composed of a plurality of visualization pixels. Just like the volume data, the visualization images can be stored.

The parameter set represents those input parameters for the image synthesis algorithm on the basis of which the image synthesis algorithm can generate one or more visualization image(s) of the volume data. In other words, the parameter set can be conceived as a control command set for the image synthesis algorithm. In other words, the parameter set can contain in code which views, perspectives, scene illuminations, transfer functions, transparencies, section planes, etc. are respectively implemented in the individual visualization images.

In particular, the parameter set can specify the generation of N different visualization images. N can be greater than or equal to 6, preferably greater than 10, more preferably greater than 20 and even more preferably greater than 50.

In 3D computer graphics, 3D modeling is the process of development of a mathematical and coordinate-based representation of any surface of an object in three dimensions. Three-dimensional (3D-) models represent a physical object, for example in that they use a collection of points in the 3D space, which are connected by different geometric units such as triangles, lines, curved surfaces, etc. Their surfaces can be defined further by texture mapping. In particular, the 3D model can have a lower storage requirement than the volume data.

The 3D model therefore represents a digital or virtual model of the object. In particular, the 3D model represents a digital or virtual view model of the object. Two-dimensional images can be generated by 3D-rendering on the basis of the 3D model. The rendering of a 3D model is often simpler than the rendering of volume data. The 3D model can therefore be continuously observed from different views by way of manipulation of the 3D model by a user.

When calculating the 3D model a three-dimensional reconstruction of the 3D model takes place from the individual visualization images, which show, in particular, different views of the object. The visualization images represent only discrete views, by way of which the 3D model can be interpolated during calculation. The calculation of 3D models from individual images is basically known.

The 3D model can be supplied, for example, as a data set. In particular, the 3D model can be stored in a storage facility and thus supplied. In particular, the 3D model can be supplied for downloading. Furthermore, the 3D model can be supplied by transfer, for example via an Internet and/or an intranet.

The 3D model supplies, on the basis of rendered volume data, a data set, which can be simultaneously portably and interactively visualized. The step of rendering the underlying volume data with the image synthesis algorithm can guarantee a high image quality for visualizations on the basis of the 3D model. At the same time the volume of data and the complexity of the generation of visualization images is reduced, and this improves the transferability and the user-friendliness for the end user. In other words, the volume data is pre-processed, compressed, optimized and encoded with regard to its three-dimensional visualization through the use of the image synthesis algorithm and calculation of the 3D model.

According to one aspect, the step of calculating the 3D model takes place by photogrammetry.

In exemplary embodiments the calculation by photogrammetry comprises extracting three-dimensional measurements from two-dimensional data (in other words, the visualization images). For example, the spacing between two points, which lie on a plane parallel to the image plane, can be determined by measuring their spacing in the visualization image since the scale is known. Furthermore, color ranges and/or color values, the variables such as albedo, mirror reflection, metallicity or ambient occlusion can be extracted for the purpose of calculation of the 3D model.

The 3D model can be easily and reliably generated from the visualization images by photogrammetry.

According to one aspect, the set has a plurality of different visualization images, which represent different views of the three-dimensional object respectively.

The 3D model can be efficiently generated by the supplying of different views.

According to one aspect, the 3D model is implemented as a surface model.

Surface model means, in other words, that the surfaces of the 3D model are covered with textures and colors and are not, for instance, transparent. An embodiment of this kind is suitable for the subsequent transfer of the 3D model and further use by an end user since the model is thus limited to essential data, which can also be easily interpreted by the end user.

According to one aspect, the method also comprises a step of supplying a requirement profile for the 3D model to be created, with the requirement profile indicating one or more properties of the 3D model to be created, with the parameter set also being ascertained on the basis of the requirement profile in the step of ascertaining the parameter set.

For example, the requirement profile can be predetermined. Furthermore, supplying the requirement profile can comprise selecting from a plurality of different selection requirement profiles. The selection requirement profiles can relate, for example, to different intended uses of the 3D model respectively. The requirement profile can relate to higher-order properties of the 3D model, which are independent of the specific data set, such as a data volume, the views of the object available in the 3D model, or a resolution of the 3D model. The requirement profile can also depend on the volume data. Low-resolution volume data thus often requires different visualization images for generating the 3D model than high-resolution volume data. Consequently the requirement profile can also be supplied on the basis of the volume data.

If the parameter set is determined on the basis of the requirement profile, suitable parameters for actuating the image synthesis algorithm can be purposefully supplied. As a result those visualization images, which result in a 3D model having the desired properties, can be automatically generated.

According to one aspect, the parameter set has a first parameterization and a second parameterization. The first parameterization relates to the (higher-order) mapping properties of the volume data (or is embodied to define them). The second parameterization is embodied for generating a plurality of different views of the three-dimensional object with the same first parameterization in order to thus generate the plurality of different visualization images of the set.

Division into first and second parameterizations means different visualization images can be generated, which show different views of the object but have the same global mapping properties, such as scene illumination, color, transparency, contrast, lighting, sharpness, etc. A coherent set of visualization images can be consequently generated, and this facilitates calculation of the 3D model.

According to one aspect, the first parameterization comprises a transfer function for mapping the volume data onto the visualization image and/or a segmenting of the three-dimensional object and/or a clipping mask.

A transfer function comprises representation parameters. They associate a particular color, transparency, contrast, lighting, sharpness and the like with each value in the volume data. Generally speaking the representation parameters influence the type of representation of objects of the corresponding object type in the visualization image output to the user.

A clipping mask can be implemented, for example, as a clipping plane or different kind of clipping area with which individual constituent parts of the object can be cut away and thus excluded from the volume rendering by the image synthesis algorithm. For example, it is thus possible to look into an object volume if an outer surface is cut away by a clipping mask.

Segmenting can comprise an identification of at least one region in the volume data, whereby this region can be treated differently in the volume rendering to the rest of the volume data outside of the region.

Use of a uniform transfer function or segmenting or clipping mask makes it possible to guarantee not only a uniform appearance of the visualization images but also to implement segmentings and/or clipping effects in the 3D model.

According to one aspect, the method comprises a step of supplying a preference of the user for volume rendering, with the parameter set being generated on the basis of the preference of the user.

The preference can comprise, for example, preferred mapping properties of an end user. This can comprise, for example, a preferred scene illumination, a particular color, transparency, contrast, lighting, sharpness, clipping area, segmenting and the like. In particular, a preference can comprise a preferred transfer function and/or a preferred first parameterization.

Taking into account the user preference makes it possible to not only take into account individual preferences of a user but for the user to co-decide which sections of the object are to be taken into account when generating the 3D model. The user can thus set, for example, a clipping plane by way of the preference in order to look into the inside of the object.

According to one aspect, supplying the preference comprises supplying a plurality of different example visualization images to the user for selection, with each of the example visualization images having been generated with different mapping properties or a different first parameterization, and receiving a selection of one of the example visualization images by the user and determining the preference on the basis of the mapping properties and/or the first parameterization of the selected example visualization images. In particular, the example visualization images can likewise be generated on the basis of the volume data using the image synthesis algorithm.

The user can consequently easily define a preference without having to concern themselves with the complex setting options of the image synthesis algorithm.

According to one aspect, the steps of supplying the volume data, of supplying the image synthesis algorithm, of ascertaining the parameter set, of generating the set and of calculating the 3D model take place in a first computing facility (or computing device). In the step of supplying, the 3D model is supplied to a second computing facility (or computing device), which second computing facility is different from the first computing facility, with the second computing facility comprising, in particular, a portable user end device.

The first computing facility can be embodied as a central or decentral computing facility. The first computing facility can be implemented, in particular as a local or Cloud-based processing server. The first computing facility can have one or more processor(s). The processors can be embodied as a central processing unit (CPU for short) and/or as a graphics processing unit (GPU for short).

The second computing facility can be embodied as a client or user client. The second computing facility can have a user interface via which an end user can interact with the 3D model and may have different views of the 3D model displayed. The user interface can have an input apparatus for this, such as a touchscreen or a computer keyboard, and an output apparatus, such as a screen. The second computing facility can have one or more processor(s). The processors can be embodied as a central processing unit (CPU for short) and/or as a graphics processing unit (GPU for short). The second computing facility can be embodied as what is known as a one-chip system (a technical term for this is “System-on-a-chip”, SoP for short), which controls all functions of a device. The computing facility can be embodied, in particular, as a portable user end device such as a laptop, tablet or a smartphone.

In other words, the comparatively complex calculation of the 3D model, including the rendering of the visualization images, can take place in a powerful computing facility. The 3D model can then be supplied to a computing facility, which has less computing power. Solely a visualization for the end user on the basis of the 3D model has to take place on the second computing facility. Owing to the lower data volume this is easier than a visualization on the basis of the volume data. The computing-intensive steps which are complicated to control by the user are thus carried out off-line in the first computing facility. Consequently the information necessary for

visualizing the volume data can be easily transferred and a simple and interactive visualization by the user is possible in the second computing facility.

According to one aspect, supplying comprises transferring the 3D model from the first to the second computing facility over the Internet.

Transfer over the Internet makes flexible supplying of the 3D model, and thus improved access, possible.

According to one aspect, the image synthesis algorithm implements a path tracing or ray casting method.

Particularly realistic visualizations can be generated by such methods, and this increases the benefit of the overall method. Said methods are complex to apply but the inventive implementation in a 3D model means said methods can be easily used by an end user and that they intrinsically deliver good visualization.

According to one aspect, the volume data is generated by a medical imaging method and represents one or more organ(s) of a patient. Furthermore, the method comprises a step of supplying patient information associated with the patient, with the parameter set being generated on the basis of the patient information.

The patient information can comprise medical information, which is associated with the patient and or the volume data of the patient. The patient information can indicate or relate to or contain, for example, one or more body part(s) and/or one or more organ(s) of the patient. The patient information can indicate or relate to or contain, for example, one or more diagnostic finding(s) of the patient or of a body part or organ of the patient, moreover. In other words, the patient information can show which aspects of the volume data are particularly relevant to the respective patient and can thus be taken into account and/or highlighted when generating the visualization images.

The parameter set, and therewith the visualization images, can be specifically adjusted to the individual application relevant to the patient by taking into account the patient information. It is thus possible to ensure that relevant sections of the volume data are also included in the 3D model.

According to one aspect, the patient information can comprise a structured or unstructured document or date, which contains one or more text element(s).

According to one aspect, the patient data comprises a medical report on diagnostic findings of the patient, and/or an electronic medical record of the patient, and/or a diagnosis task for creation of a diagnostic finding for the patient by a user.

Said patient information can denote, in particular, body parts and/or organs of the patient, which are represented by the volume data. The entry in said patient information can accordingly indicate that these body parts and/or organs are relevant to the 3D model and should be taken into account accordingly.

According to one aspect, the 3D model is supplied in the step of supplying as further patient information and, in particular, as part of an (electronic) medical report on diagnostic findings for the patient.

The 3D model is consequently associated with the patient and archived and is available for subsequent use.

According to one aspect, the method also comprises a step of determining at least one selected organ for representation in the 3D model on the basis of the patient information, and a step of generating a segmenting mask for the volume data for segmenting the at least one selected organ, with the plurality of different visualization images also being generated on the basis of the segmenting mask in the step of generating.

In particular, details of the at least one selected organ can be extracted from the patient information. For example, known computer linguistics algorithms can be used for this, which are embodied to identify text elements contained in the patient information and attribute them a meaning.

In other words, the volume data can be automatically segmented by way of an evaluation of the patient information. Calculation of the 3D model targeted at the individual medical application can consequently take place.

According to one aspect, the patient information has an annotation based on the three-dimensional object, with the annotation being inserted in the 3D model during the step of generating.

The annotation can refer, for example, to at least one body part and/or organ of the patient, which are represented in the volume data. The annotation can comprise, for example, a measurement and/or a note, which was applied to the volume data. For example, the annotation can be inserted in the 3D model in close proximity to the respective body part and/or organ. Alternatively, the annotation can be inserted in the visualization images.

The 3D model can consequently be augmented with additional information, which is then supplied together with the 3D model.

According to one aspect, the 3D model is supplied in a viewing application, which makes it possible for the user to select and look at different views of the 3D model.

For example, the viewing application can be embodied to calculate two-dimensional visualization images of the 3D model. Furthermore, the viewing application can be embodied to receive a user input directed toward the selection of a view of the 3D model and supply the selected view as the visualization image of the 3D model on the basis of the user input. In particular, the viewing application can be embodied to run in the second computing facility.

Supplying the 3D model as a viewing application enables easy transfer of the 3D model with interactive operation.

According to one aspect, the viewing application is implemented as a Web application. This has the advantage of easy access and a lightweight implementation of the viewing application.

According to one aspect, in the step of supplying the 3D model, the 3D model can be supplied in a storage facility for downloading.

For example, the storage facility can be embodied as Cloud storage. Alternatively, the storage facility can be embodied as a local storage device. Supplied for downloading can mean, in particular, that the 3D model is made accessible in the storage facility for a user.

For example, the step of supplying can comprise generating a link to direct access to the 3D model supplied in the storage facility for downloading, and supplying the link. In particular, the link can be supplied in the viewing application or be retrievable via this application. Both easy transfer of the 3D model and good accessibility for a user can be achieved in this way.

According to one aspect, the method also comprises a step of supplying a trained function, which is embodied to ascertain on the basis of volume data (and optionally patient information and/or a requirement profile and/or a user preference) a parameter set for actuating an image synthesis algorithm, which parameter set is suitable for generating, with the image synthesis algorithm, a set of a plurality of different visualization images on the basis of the volume data set, with the parameter set being ascertained in the step of ascertaining the parameter set by applying the trained function (to the volume data and optionally patient information and/or a requirement profile and/or a user preference).

A trained function generally maps input data onto output data. The output data can, in particular, still depend on one or more parameter(s) of the trained function here. The one or more parameter(s) of the trained function can be determined and/or adjusted by training. Determining and/or adjusting the one parameter or the plurality of parameters of the trained function can be based, in particular, on a pair of training input data and associated training output data, with the trained function being applied to the training input data to generate training mapping data. In particular, determination and/or adjustment can be based on a comparison of the training mapping data and the training output data. In general, a trainable function, in other words, a function with parameters that have not yet been adjusted, is also referred to as a trained function.

Other terms for trained function are trained mapping rule, mapping rule with trained parameters, function with trained parameters, algorithm based on artificial intelligence, machine learning algorithm. One example of a trained function is an artificial neural network. Instead of the term “neural network”, the term “neural net” can also be used. Basically a neural network has a structure like a biological neural net such as a human brain. In particular, an artificial neural network comprises an input layer and an output layer. It can also comprise a plurality of layers between input and output layer. Each layer comprises at least one, preferably a plurality of node(s). Each node can be taken to mean a biological processing unit, for example a neuron. In other words, each neuron corresponds to an operation, which is applied to input data. Nodes of one layer can be connected to nodes of other layers by edges or connections, in particular by directed edges or connections. These edges or connections define the data flow between the nodes of the network. The edges or connections are associated with a parameter, which is frequently referred to as a “weight” or “edge weight”. This parameter can regulate the importance of the output of a first node for the input of a second node, with the first node and the second node being connected by an edge.

In particular, a neural network can be trained. In particular, training of a neural network is carried out on the basis of the training input data and associated training output data in accordance with a “supervised” learning technique (“supervised learning”), with the known training input data being input into the neural network and the output data generated by the network being compared with the associated training output data. The artificial neural network learns and adjusts the edge weights for the individual nodes independently as long as the output data of the last network layer does not sufficiently correspond to the training output data.

In particular, a trained function can also be a deep artificial neural network (or “deep neural network”). According to some implementations, the trained function has a neural network and, in particular, a convolutional neural network. In particular, the convolutional neural network can be embodied as a deep convolutional neural network. The neural network has one or more convolutional layer(s) and one or more deconvolutional layer(s). In particular, the neural network can comprise a pooling layer. The use of convolutional layers and/or deconvolutional layers means a neural network can be used particularly efficiently for deriving a parameter set since, despite many connections between node layers, only a few edge weights (namely the edge weights corresponding to the values of the convolutional core) have to be determined. The accuracy of the neural network can therewith also be improved with an identical number of training data. In particular, it has been found that convolutional neural networks can effectively process volume data as input data.

For example, a data set for training the trained function can comprise training input data and training output data. Training input data comprises volume data and possibly patient information and/or a requirement profile and/or a user preference. Training output data comprises a (verified) training parameter set for input into the image synthesis algorithm and for generating a plurality of visualization images. The training parameter set can be supplied, for example, by an expert, who is entrusted with both image synthesis algorithms and with the demands on the visualization images for calculation of the 3D model.

The parameter set can be efficiently automatically generated by the use of a trained function. Compared to a rule-based generation, the use of a trained function has the advantage that the training renders the trained function capable of creating a dynamic adjustment to different circumstances.

According to one aspect, a method for supplying a trained function is provided, which is embodied to supply a parameter set with which an image synthesis algorithm for generating a 3D model can be actuated. The method has a plurality of steps. A first step is directed toward supplying training input data, with the training input data having volume data (and optionally patient information and/or a requirement profile and/or a user preference) representing a three-dimensional object. A further step is directed toward supplying training output data, with the training output data having a training parameter set, which training parameter set is suitable for generating, with the image synthesis algorithm, a set of a plurality of different visualization images on the basis of the volume data. A further step is directed toward generating a parameter set by applying the trained function to the volume data (and optionally patient information and/or a requirement profile and/or a user preference), with the parameter set being suitable for generating, with the image synthesis algorithm, a set of a plurality of different visualization images on the basis of the volume data. A further step is directed towards comparing the parameter set with the training parameter set. A further step is directed toward adjusting the trained function on the basis of the comparison.

According to a further aspect, a training system for supplying a trained function is provided, which is embodied to carry out one or more method step(s) of said method for supplying a trained function.

According to one aspect, an apparatus for supplying a 3D model of a three-dimensional object represented by volume data is supplied. The apparatus has a computing facility and an interface. The interface is embodied to receive the volume data and to supply the 3D model. The computing facility is embodied to host an image synthesis algorithm, which image synthesis algorithm is embodied for visualization of the three-dimensional object by mapping the volume data onto visualization pixels of a two-dimensional visualization image.

The computing facility is also embodied to ascertain a parameter set for actuating the image synthesis algorithm, which parameter set is suitable for generating, with the image synthesis algorithm, a set of a plurality of different visualization images on the basis of the volume data set. The computing facility is also embodied to generate the set of the plurality of different visualization images by mapping the volume data, with the image synthesis algorithm, using the parameter set. The computing facility is also embodied to calculate the 3D model on the basis of the set of the plurality of visualization images.

In particular, the computing facility can correspond to the second computing facility described herein.

For example, the computing facility can comprise a configuration module, which is embodied to supply the parameter set on the basis of the volume data (and optionally on the basis of patient information and/or a requirement profile and/or a user preference). The configuration module can be embodied to carry out the trained function.

For example, the computing facility can comprise a visualization module, which is embodied to generate the visualization images on the basis of the parameter set and the volume data. The visualization module can be embodied to run the image synthesis algorithm.

For example, the computing facility can comprise a modeling module, which is embodied to generate the 3D model on the basis of the visualization images. The modeling module can be embodied to carry out a photogrammetry algorithm.

The interface can be embodied in general for data exchange between the computing facility and further components. The interface can be implemented in the form of one or more individual data interface(s), which can have a hardware and/or software interface, for example a PCI bus, a USB interface, a FireWire interface, a ZigBee interface or a Bluetooth interface. The interface can also have an interface of a communications network, it being possible for the communications network to have a Local Area Network (LAN), for example an Intranet, or a Wide Area Network (WAN) or an Internet. Accordingly, the one more data interface(s) can have a LAN interface or a Wireless LAN interface (WLAN or Wi-Fi).

According to one aspect, the apparatus also has a storage facility, which is embodied to store volume data and supply it via the interface.

The storage facility can be, in particular, part of what is known as a Picture Archiving and Communication system (PACS). In addition or alternatively, the storage facility can be part of a medical information system, such as a hospital or laboratory information system.

The advantages of the proposed apparatus substantially correspond to the advantages of the proposed method. Features, advantages or alternative embodiments can likewise be transferred to the other claimed subject matters, and vice versa.

According to one aspect, a computer program product having a computer program is supplied, which can be directly loaded into a storage device of an apparatus, having program segments in order to carry out all steps of the method for supplying a 3D model or for supplying a trained function according to one of the aspects described herein when the program segments are executed by the apparatus.

According to one aspect, a computer-readable storage medium is supplied on which program segments, which can be read and executed by an apparatus, are stored in order to carry out all steps of the method for supplying a 3D model or for supplying a trained function according to one of the aspects described herein when the program segments are executed by the apparatus.

The computer program products can comprise software having a source code, which still has to be compiled and linked or which only has to be interpreted, or an executable software code, which for execution merely still has to be loaded in the processing unit. The method can be carried out quickly, in an identically repeatable manner and robustly by the computer program products. The computer program products are configured such that they can carry out the inventive method steps via the computing unit. The computing unit has to have the requirements, such as an appropriate working memory, an appropriate processor, an appropriate graphics card or an appropriate logic unit, so the respective method steps can be efficiently carried out.

The computer program products are stored, for example, on a computer-readable storage medium or saved on a network or server from where they can be loaded into processor of the respective computing unit, which can be directly connected to the computing unit or can be embodied as part of the computing unit. Furthermore, control information of the computer program products can be stored on a computer-readable storage medium. The control information of the computer-readable storage medium can be embodied in such a way that it carries out an inventive method when the data carrier is used in a computing unit. Examples of computer-readable storage medium are a DVD, a magnetic tape or a USB stick on which electronically readable control information, in particular software, is stored. All inventive embodiments of the previously described method can be carried out when this control information is read from the data carrier and stored in a computing unit. Embodiments of the present invention can thus also start from said computer-readable medium and/or said computer-readable storage medium.

The advantages of the proposed computer program products or the associated computer-readable media substantially correspond to the advantages of the proposed method.

### BRIEF DESCRIPTION OF THE DRAWINGS

Further characteristics and advantages of the present invention will become obvious from the explanations of exemplary embodiments below with reference to schematic drawings. Modifications mentioned in this connection can be combined with each other respectively to embody new embodiments. Identical reference characters are used in different figures for identical features.

In the drawings:

FIG. 1 shows a schematic representation of a first embodiment of a system for supplying a 3D model,

FIG. 2 shows a flowchart of a method for supplying a 3D model according to one embodiment,

FIG. 3 shows a data flowchart of a method for supplying a 3D model according to one embodiment,

FIG. 4 shows a trained function for generating a parameter set for input into an image synthesis algorithm,

FIG. 5 shows a schematic representation of an embodiment of a system for supplying the trained function, and

FIG. 6 shows a flowchart of a method for supplying a trained function for generating a parameter set.

### DETAILED DESCRIPTION

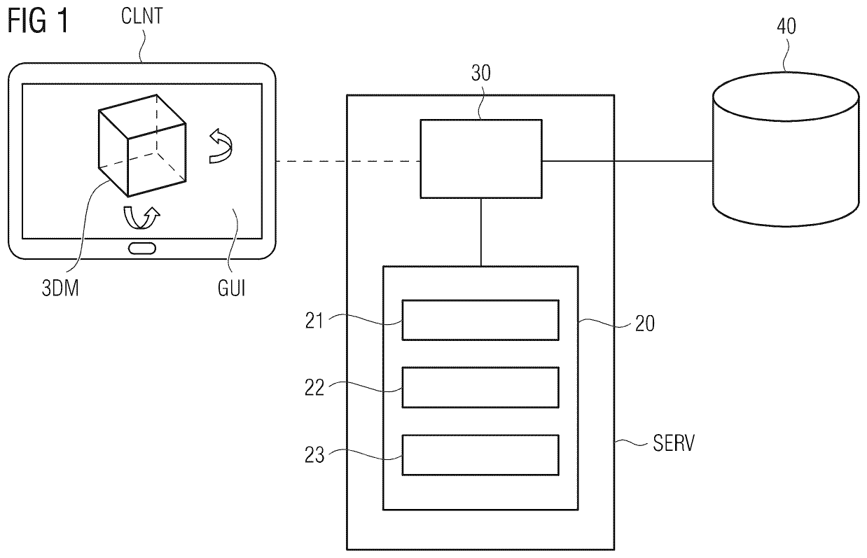

FIG. 1 shows a system 1 for supplying a 3D model of an object according to one embodiment. The system 1 has a server SERV (as an example of a second computing unit), an interface 30, a user end device or client CLNT (as an example of a first computing facility) and a storage facility 40. The server SERV is basically embodiment for calculation of a 3D model of a three-dimensional object on the basis of volume data VD describing the three-dimensional object. The volume data VD can be supplied to the server SERV from the storage facility 40 via the interface 30.

The storage facility 40 can be embodied as a central or decentral database. The storage facility 40 can be, in particular, part of a server system. In the medical field the storage facility 40 can be, in particular, part of a medical information system such as a hospital information system (or HIS for short) and/or a PACS system (PACS stands for picture archiving and communication system) and/or a laboratory information system (LIS). The storage facility 40 can also be embodied as what is known as Cloud storage.

The volume data VD of the three-dimensional object can be stored in the storage facility 40. The volume data VD represents the three-dimensional object. The volume data VD has a three-dimensional data set comprising a plurality of volume pixels, what are known as voxels. One or more geometric and/or physical properties of the three-dimensional object can be encoded in a spatially resolved manner in the volume data VD. For example, the voxel values can represent a measure of the local density of the three-dimensional object at the location of the voxel. The volume data VD can have been generated by a corresponding imaging method, for example by radiography or computed tomography methods.

In medical applications the volume data VD can have been generated by a medical imaging method. The volume data VD can then represent a body part of a patient and show, for example, one or more organ(s) of the patient. For example, the volume data VD can have been generated by radiography, computed tomography (CT), magnetic resonance tomography (MR), ultrasound and/or positron emission tomography (PET). The volume data VD can be formatted, for example, in the DICOM format. DICOM stands for Digital Imaging and Communications in Medicine and denotes an open standard for the storage and exchange of information in medical image data management.

Supplying the volume data VD can comprise, for example, loading the volume data VD into a working memory (not shown) of the server SERV.

In addition, further information can be present in the system 1, which is relevant to the generation of the three-dimensional model 3DM. These are, for example, specific requirement profiles AP for the generation of the 3D model 3DM, certain user preferences NP with regard to the generation of the 3D model, or further information relating to the object for modelling. In the medical application the latter can be, for example, patient information PI. Additional information can be stored in the storage facility 40 (or the medical information system) or be supplied by a user of the user terminal CLNT.

The server SERV is embodied to calculate a three-dimensional model 3DM of the object shown in the volume data VD on the basis of the volume data VD. The server SERV be embodied to take into account the additional information. The server SERV can have one or more processor(s). The processors can be implemented as a Central Processing Unit (CPU), a Graphics Processing Unit (GPU), a digital signal processor (DSP), an image processing processor, an integrated (digital or analog) circuit or combinations of said components. The server SERV can be implemented as individual components or have a plurality of components, which operate in parallel or in series. Alternatively, the server SERV can have a real or virtual group of computers, such as a cluster or a Cloud. Depending on the embodiment the server SERV can be embodied as a local server or as a Cloud server. The server SERV is embodied, for example, by way of computer-readable instructions, by design and/or hardware in such a way that it can carry out one or more method step(s) according to embodiments of the present invention.

The server SERV is embodied, in particular, to not calculate the 3D model 3DM directly from the volume data VD but by way of an intermediate step. This intermediate step provides firstly generating a set S of two-dimensional visualization images VB from the volume data VD. For this purpose a physical renderer method is preferably used, which can be based on the simulation of optical beam paths. Very realistic mappings of the volume data VD can be generated as a result. The server SERV is also embodied to then calculate the 3D model 3DM from these images. Compared to a direct calculation of the 3D model 3DM from the volume data VD this has the advantage that mapping effects such as transparency, shading, color bleeding, occlusion effects, etc. can be taken into account. It is precisely in medical image data that an expedient calculation of a 3D model directly from the volume data VD is often not readily possible since different tissues are not resolved selectively enough by medical imaging methods and first have to be carved out by a rendering method.

To a certain extent the 3D model represents a data set that is reduced or compressed compared to the volume data VD, which contains the fundamental properties for viewing the object. This can be, for example, a collection of points in the 3D space, which are connected by different geometric units such as triangles, lines, curved surfaces, etc. and thus characterize the surfaces relevant to the observer. In contrast, an inner structure of the object, for example, can be omitted.

The server SERV or its computing unit 20 can have different modules 21, 22, and 23 to supply the 3D model 3DM on the basis of the input data. The undertaken division of the computing unit 20 into modules 21-23 serves merely to simplify the explanation of the mode of operation of the computing unit 20 or of the server SERV and should not be understood as limiting. The modules 21-23 or their functions can also be combined in a unit. The modules 21-23 can also be conceived, in particular, as computer program products or computer program segments, which implement one or more of the method step(s) described below when executed in the computing unit 20 or the server SERV.

The module 21 can be conceived as a configuration module 21. The configuration module 21 can implement, host or control a configuration algorithm KA, which is embodied to ascertain on the basis of the input data, i.e. the volume data VD and possibly additional information, a parameter set PS for controlling an image synthesis algorithm BSA. The parameter set PS contains details, which cause the image synthesis algorithm BSA to output the set S of visualization images VB. According to some embodiments, the configuration algorithm KA can have a trained function.

The module 22 can be conceived as a visualization module 22 or “volume rendering engine”. The visualization module 22 can implement, host or control an image synthesis algorithm BSA, which is embodied to map the volume data VD onto the visualization image VB or the pixels of the visualization image VB. For calculation of the mapping the image synthesis algorithm BSA can, for its part, have different modules. For example, the image synthesis algorithm BSA can comprise a ray casting module in which the pixels of the visualization images VB are calculated with the method of ray casting. Furthermore, the image synthesis algorithm BSA can have a path tracing module in which the pixels of the visualization images VB are calculated according to the method of path tracing. In addition, individual modules of the image synthesis algorithm BSA can relate to supplementary mapping effects. These supplementary mapping effects can comprise, in particular in ray casting methods, for example effects of ambient occlusion, shadow effects, translucence effects, color bleeding effects, surface shadows, complex camera effects and/or lighting effects due to any ambient lighting conditions.

The module 23 can be conceived as a modeling module 23. The modeling module 23 is embodied to calculate a 3D model from visualization images VB calculated by the image synthesis algorithm BSA. The modeling module 23 can implement, host or control a modelling algorithm MA, which is embodied to calculate the 3D model 3DM from the visualization images VB. In particular, the modelling algorithm MA can implement a photogrammetry method.

Once the 3D model 3DM has been calculated it should be displayed to the user. For this purpose the 3D model 3DM is supplied to the user end device CLNT via the interface 30. The user end device CLNT can have an output facility like a screen, which for displaying a graphical user interface GUI is configured for the representation of different views of the 3D model. In addition, the user end device CLNT can have an input facility like a touchscreen, with which the user can select different views of the 3D model. The user end device CLNT can have a computing unit, such as a processor, which is embodied to calculate two-dimensional visualizations on the basis of the selected views and the 3D model 3DM. In exemplary embodiments the user end device CLNT is a smartphone or a tablet PC or a laptop or a desktop PC.

The interface 30 can have one or more individual data interface(s), which guarantee data exchange between the components SERV, CLNT, 40 of the system 1. The one or more data interface(s) can have a hardware and/or software interface, for example a PCI bus, a USB interface, a FireWire interface, a ZigBee interface or a Bluetooth interface. The one or more data interface(s) can have an interface of a communications network, wherein the communications network can have a Local Area Network (LAN), for example an Intranet, or a Wide Area Network (WAN) or an Internet. The one or more data interface(s) can accordingly have a LAN interface or a Wireless LAN interface (WLAN or Wi-Fi). In particular, the interface between user end device CLNT and server SERV is an Internet interface and the data is transferred between user end device CLNT and server SERV over the Internet.

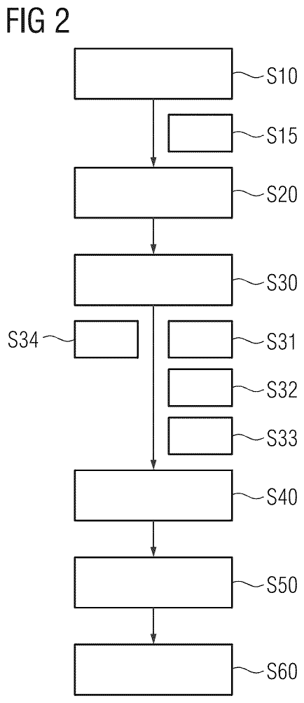

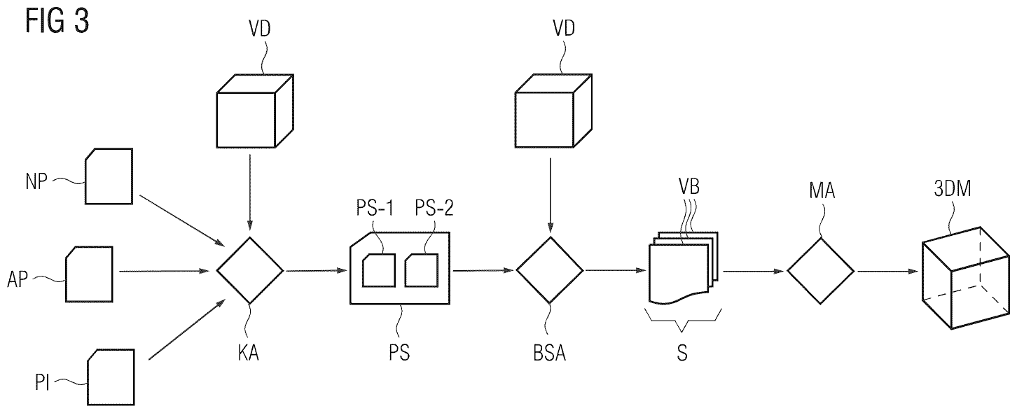

FIG. 2 represents a schematic flowchart of a method for visualizing a three-dimensional object. The order of the method steps is limited by neither the represented sequence nor by the selected numbering. The order of the steps can thus possibly be interchanged and individual steps can be omitted. FIG. 3 schematically represents an associated data flowchart. FIGS. 2 and 3 represent the method by way of example using medical image data as the volume data VD. It is understood that the method can largely also be applied to any other types of image data.

A first step S10 is directed toward supplying the volume data VD. Supplying can be achieved by a retrieval of the volume data VD from the storage facility 40 and/or loading of the volume data VD into the server SERV. The volume data VD represents a three-dimensional object. In the discussed example this is a body part of a patient.

In an optional sub-step S15 additional information can be supplied for targeted generation of the set S of visualization images VB and therewith for targeted calculation of the 3D model 3DM. The additional information can comprise, for example, patient information PI, a requirement profile AP and/or a user preference NP.

The patient information PI can comprise, for example, a medical report on diagnostic findings of the patient, and/or an electronic medical record of the patient and/or a diagnosis task for creation of a diagnostic finding for the patient by a user. The patient information PI can therewith contain details, which are relevant to the visualization and model generation, such as a detail of a body part to be diagnosed, one or more suspected diagnoses relating to one or more body part(s), a detail of a medical finding, etc. In particular, the patient information PI can comprise one or more structured or unstructured text document(s), which contain natural language. Such information can be supplied by or downloaded from the storage facility 40.

Requirement profiles AP can in general comprise details about which different individual images are necessary for generating a particular 3D model 3DM. This can comprise the number of visualization images VB or a detail of the views to be mapped. Different types of models 3DM can have different requirement profiles AP respectively. When selecting a model 3DM the associated requirement profile AP can consequently be selected and supplied. Requirement profiles AP can be stored in the server SERV or be supplied by or downloaded from the storage facility 40.

User preferences NP can in general comprise preferred user settings for the calculation of the 3D model 3DM, such as mapping parameters (scene illumination, resolution, available views, transfer functions, etc.) or the type of 3D model 3DM desired by the user. User preferences NP can be supplied, for example, by the user end device CLNT. Alternatively, user preferences NP can be stored in the server SERV or be supplied by or downloaded from the storage facility 40.

A further step S20 is directed toward supplying the image synthesis algorithm BSA. Supplying can be achieved by provision and/or retrieval of the image synthesis algorithm BSA in or from any storage device (for example a storage facility 40 of a medical information system or any storage device of the server SERV) and/or loading of the image synthesis algorithm BSA into the computing unit 20 of the server SERV.

In a further step S30 a parameter set PS is ascertained on the basis of the available input data, i.e. the volume data VD and optionally the patient information PI, the user preference NP and/or the requirement profile AP. The parameter set PS is suitable for generating, with the image synthesis algorithm BSA, the set S of visualization images VB from which a 3D model 3DM can be calculated.

The parameter set PS can accordingly be conceived as a set of control commands with which the image synthesis algorithm BSA can be actuated for generating the set S of visualization images VB. Appropriate settings for the image synthesis algorithm BSA can consequently be encoded in the parameter set PS. According to exemplary embodiments, the parameter set PS has two different types of parameter. On the one hand these are “global” parameters, which apply to all visualization images VB of a set S. This relates to, for example, the scene illumination, the transfer function, the contrast, the sharpness, a transparency of one or more region(s) of the volume data VD, a section plane through the volume data VD, one or more item(s) of additional information for fading into the visualization images VB, etc. These “global” parameters are combined in the first parameterization PS-1. On the other hand, the parameter set PS also comprises a second parameterization PS-2 in which instructions for generating the different views or perspectives for the subsequent calculation of the 3D model 3DM are encoded.

Optionally, the 3D model 3DM to be calculated can be defined in a sub-step S31. This can take place automatically on the basis of the volume data VD or the optional patient information PI and/or the optional requirement profile AP. It is thus possible to define, for example, that a particular 3D model is to be generated for particular patient information PI (for example a particular finding to be created). The 3D model to be generated can be automatically selected in step S31 from a plurality of different predetermined 3D models. Alternatively, a particular 3D model 3DM can be selected by a user of the end terminal CLNT and be transferred to the server SERV, for example via the user preference NP.

Optionally, the volume data VD can be automatically segmented in step S30 for the visualization and therewith the 3D model 3DM. For this purpose firstly one or more selected organ(s) can be identified in an optional sub-step S32 for representation in the 3D model 3DM. This can take place, for example, on the basis of the patient information PI. If, for example, the lungs are mentioned in the patient information PI, it is possible to infer the lungs as the selected organ from this. The patient information PI can be analyzed for this, for example with a computer linguistics algorithm, which is embodied to detect one or more keyword(s) or terms in the patient information PI. In a further sub-step S33 one or more segmenting mask(s) can be ascertained for application to the volume data VD. The segmenting mask(s) are embodied to segment, in other words, identify, the image data pertaining to the selected organ in the volume data VD.

As mentioned, the parameter set PS can be ascertained by an appropriately adjusted configuration algorithm KA on the basis of the input data (volume data VD or optionally patient information PI, user preference NP and/or requirement profile AP). The configuration algorithm KA can comprise a computer linguistics algorithm, which is embodied for algorithmic processing of natural language in the form of text or speech data. Furthermore, the configuration algorithm KA can comprise a trained function TF, which is embodied to generate the parameter set PS on the basis of the input data. A trained function of this kind can be supplied accordingly in an optional step S34.

In step S40 the set S of visualization images VB is generated on the basis of the parameter set PS and the volume data VD. For this purpose the volume data VD and the parameter set PS are input into the image synthesis algorithm BSA. Optionally, a segmenting mask ascertained in step S30 can be taken into account.

In step S50 the visualization images VD generated in step S40 are used to calculate 3D model 3DM of the object shown in the volume data VD. For this purpose the visualization images VB are input into a modelling algorithm MA, which is embodied for calculation of a 3D model of an object on the basis of different two-dimensional views. In other words, modelling algorithm MA converts a plurality of two-dimensional visualization images VB into the three-dimensional model 3DM.

In particular, the modelling algorithm MA can implement a method of photogrammetry. The modelling algorithm MA is embodied to determine the position and shape of the three-dimensional object from the set S of visualization images VB by image measurement. For this purpose the mapping geometry at the instant of calculation of the visualization images VB can be restored. This restoration can take place, for example, in accordance with the laws of central projection while adhering to the coplanarity. Each image defines a direction for the object point for a mapped point together with the center of projection of the respective virtual camera. With known orientation of the virtual camera and known mapping geometry it is then possible to describe each ray in the space. By using at least two homologous (corresponding) image points of two different recording positions the modelling algorithm MA, with knowledge of the mutual position (relative orientation), can cause the two rays to intersect and thus three-dimensionally calculate each object point.

The 3D model 3DM is finally supplied in step S60. For this purpose it can be transmitted to the user end device CLNT or supplied for downloading by the user end device CLNT. The 3D model 3DM can be supplied for downloading, for example, from an appropriate storage facility 40 of the medical information system, such as Cloud storage.

Furthermore, the 3D model 3DM can be supplied with a viewing application. The viewing application can supply a graphical user interface GUI. The user can, for example, select different views of the 3D model 3DM and have them displayed via the graphical user interface GUI. The views therefore represent (similar to the visualization images VB) two-dimensional “renderings” of three-dimensional output data.

The viewing application can be activated, for example, on the user end device CLNT in that the user downloads it from an App Store and/or executes it locally. Alternatively, the viewing application can be embodied as web application, which is hosted by the server and/or the medical information system.

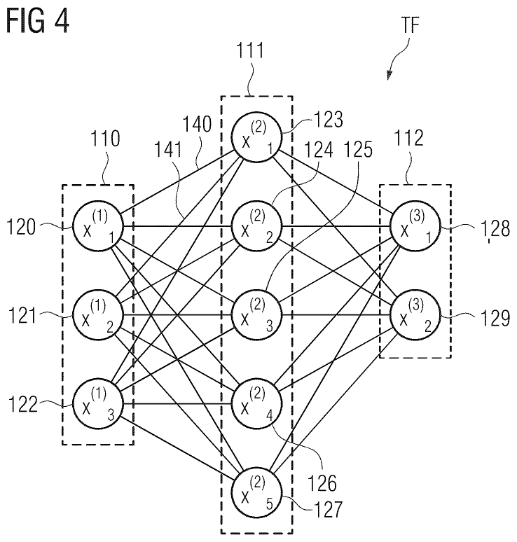

FIG. 4 shows an exemplary representation of a trained function TF, as can be used in step S30 for ascertaining a parameter set PS. The trained function TF receives the volume data VB or optionally the patient information PI, the user preference NP and/or the requirement profile AP as an input and outputs the parameter set PS, in other words, control commands for actuating the visualization module 22 or image synthesis algorithm BSA, as an output.

In the exemplary embodiment shown the trained function TF is embodied as a neural net. The neural net can also be referred to as an artificial neural net, artificial neural network or neural network.

The neural net 100 comprises nodes 120, . . . , 129 and edges 140,141, wherein each edge 140,141 is a directed connection from a first node 120, . . . , 129 to a second node 120, . . . , 129. In general, the first node 120, . . . , 129 and the second node 120, . . . ,129 are different nodes. It is also possible that the first node 120, . . . , 129 and the second node 120, . . . , 129 are identical. An edge 140,141 from a first node 120, . . . ,129 to a second node 120, . . . ,129 can also be referred to as an incoming edge for the second node and as an outgoing edge for the first node 120, . . . ,129.

The neural net 100 responds to input values x(1)1, x(1)2, x(1)3 relating to a large number of input nodes 120, 121, 122 of the input layer 110. The input values x(1)1, x(1)2, x(1)3 are applied to generate one or a large number of output(s) x(3)1, x(3)2. The node 120 is connected to the node 123, for example by an edge 140. The node 121 is connected to the node 123, for example by the edge 141.

In this exemplary embodiment the neural net 100 learns in that it adjusts the weighting factors wi,j (weights) of the individual nodes on the basis of training data. Possible input values x(1)1, x(1)2, x(1)3 of the input nodes 120,121,122 can be, for example, the individual field variables \tilde{E}_{BC}, \tilde{H}_{BC},\tilde{E}ᵢ, \tilde{H}ᵢ and/or examination information UI (if applicable).

The neural net 100 weights the input values of the input layer 110 on the basis of the learning process. The output values of the output layer 112 of the neural net 100 preferably correspond to field information FI, on the basis of which the electric and/or magnetic field forming the basis of the signature S may be at least partially suppressed. The output can take place via a single or a large number of output node(s) x(3)1, x(3)2 in the output layer 112.

The artificial neural net 100 preferably comprises a hidden layer 111, which comprises a large number of nodes x(2)1, x(2)2, x(2)3. A plurality of hidden layers can be provided, with a hidden layer using output values of another hidden layer as the input values. The nodes of a hidden layer 111 perform mathematical operations. An output value of a node x(2)1, x(2)2, x(2)3 corresponds to a non-linear function f of its input values x(1)1, x(1)2, x(1)3 and the weighting factors wi,j. After receiving input values x(1)1, x(1)2, x(1)3 a node x(2)1, x(2)2, x(2)3 totals a multiplication of each input value x(1)1, x(1)2, x(1)3, weighted by the weighting factors wi,j, as determined by the following function:

xⱼ⁽ⁿ⁺¹⁾=f(Σᵢxᵢ⁽ⁿ⁾·w_{i,j}^{(m,n)}).

The weighting factor wi,j can [be], in particular, a real number, in particular can lie in the interval of [−1; 1] or [0; 1]. The weighting factor w_{i,j}^{(m,n)} denotes the weight of the edge between the ith node or an mth layer 110,11,112 and a jth node of the nth layer 110,111,112.

In particular, an output value of a node x(2)1, x(2)2, x(2)3 is formed as a function f of a node activation, for example a sigmoidal function or a linear ramp function. The output values x(2)1, x(2)2, x(2)3 are transferred to the output node(s) 128,129. A weighted multiplication of each output value x(2)1, x(2)2, x(2)3 is totaled again as a function of the node activation f and the output values x(3)1, x(3)2 calculated therewith.

The neural net TF shows here is a feedforward neural net in which all nodes 111 process the output values of a previous layer in the form of their weighted sum as input values. Of course it is inventively possible for other neural net types to also be used, for example feedback nets, in which an input value of a node can simultaneously also be its output value.

The neural net TF can be trained via a method of supervised learning to supply the field information FI. A known procedure is back propagation, which can be applied to all exemplary embodiments of the present invention. During training the neural net TF is applied to training input data or values and has to generate corresponding, previously known training output data or values. Mean square errors (“MSE”) are iteratively calculated between calculated and expected output values and individual weighting factors are adjusted until the deviation between calculated and expected output values lies below a predetermined threshold.

Parameter sets PS, for example, which were created by visualization experts for a particular medical context and for particular volume data VD, can be accessed for supplying training data.

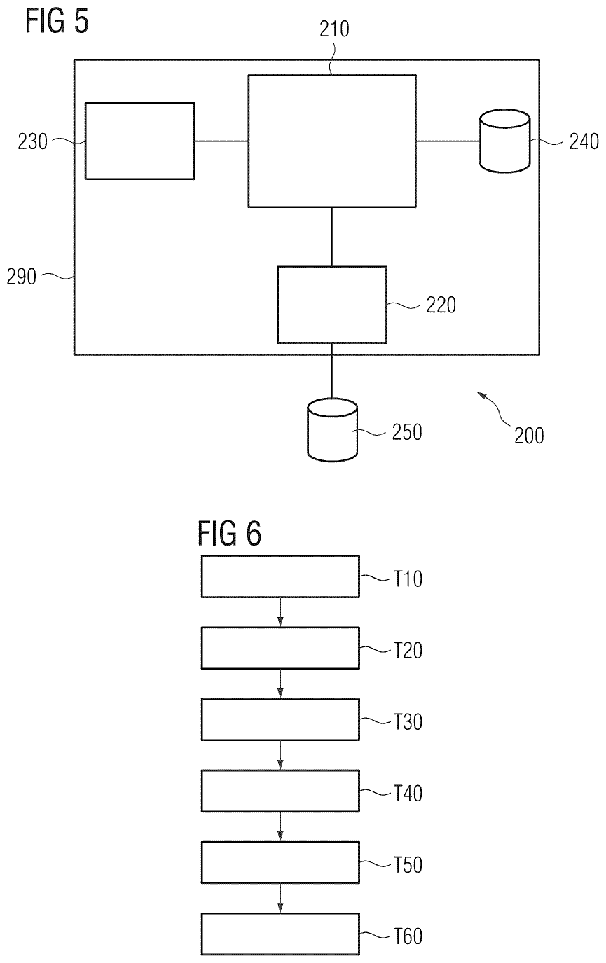

FIG. 5 shows an embodiment of a system 200 for training or supplying the trained function TF. The system 200 comprises a processor 210, an interface 220, a working memory 230, a storage facility 240 and a database 250. The processor 210, the interface 220, the working memory 230 and the storage facility 240 can be embodied as a computer 290. The processor 210 controls the operation of the computer 290 during training of the trained function TF. In particular, the processor 210 can be embodied in such a way that it carries out the method steps represented in FIG. 6. The instructions can be stored in the working memory 230 or in the storage facility 240 and/or be loaded into the working memory 230 when execution of the instructions is desired. The storage facility 240 can be embodied as a local storage device or a remote storage device, which can be accessed over a network. The method steps represented in FIG. 6 can be defined by computer program products, which are stored in the working memory 230 and/or the storage facility 240.

The database 250 can be implemented as Cloud storage or a local storage device, which is connected to the computer 290 via a wireless or wired interface 220. The database 250 can also be, in particular, part of the computer 290. The database 250 serves as an archive for the (training) volume data, training additional information (patient information PI, user preference NP and/or requirement profile AP) and/or associated training parameter sets. Furthermore, the database 250 can serve as an archive for one or more trained function(s) TF.

FIG. 6 represents a schematic flowchart of a method for supplying a trained function TF for supplying a parameter set PS. The order of the method steps is limited neither by the represented sequence nor by the selected numbering. The order of the steps can thus possibly be interchanged and individual steps can be omitted.

A first step T10 is directed toward supplying a trained function TF. The trained function TF can be supplied to the processor 210 from the database 250 via the interface 220. The trained function TF can already be pre-trained, in other words, one or more parameter(s) of the trained function TF has/have already been adjusted by the described training method and/or another training method. Alternatively, it is possible for one or more parameter(s) of the trained function to have not yet been adjusted via training data, in particular one or more parameter(s) can be preassigned by a constant value and/or by a random value. In particular, it is possible for not all parameters of the trained function TF to have not yet been adjusted via training data, in particular all parameters can be preassigned by a constant value and/or by a random value.

A second step T20 is directed toward supplying training input data. Since in use the trained function TF is to supply a parameter set PS consistent with the respective volume data VD and optionally on the basis of the associated patient information PI, the associated user preference NP and/or the associated requirement profile AP, suitable training input data is precisely training volume data and such optional additional information.

Step T30 is directed toward supplying training output data. The training output data are training parameter sets PS. A training parameter set PS represents the parameter set PS that is expedient for a set of training volume data VD, which results in a set S of visualization images VB suitable for the creation of a 3D model 3DM. The training parameter sets PS can be supplied, for example, by an expert.

In a next step T40 the training input data, i.e. training volume data VD and possibly the additional information PI, NP, AP, is input into the trained function TF. On this basis the trained function TF calculates a parameter set PS for actuating an image synthesis algorithm BSA.

In a next step T50 the parameter set PS calculated in this way is compared with the associated training parameter set PS. The trained function TF is then adjusted in step T60 on the basis of this comparison. This can occur, for example, on the basis of a cost functional, which penalizes deviations of the calculated parameter set PS from the associated training parameter set PS. One or more parameter(s) of the trained function TF can then be adjusted, in particular, such that the cost functional is minimized, for example via a back propagation. In one embodiment the cost functional can be based on a pair-wise difference of control or visualization parameters of the calculated parameter set PS and the associated training parameter set PS, for example on the total of the deviations squared. To minimize the cost functional the comparison is carried out for different pair-wise sets from the calculated parameter set PS and associated training parameter set PS until a local minimum of the cost functional is reached and the trained function TF operates satisfactorily.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “on,” “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” on, connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

In addition, or alternative, to that discussed above, units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C #, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Where it has not yet explicitly occurred but expedient and within the meaning of the present invention, individual exemplary embodiments, individual partial aspects or features thereof can be combined with each other or replaced without departing from the scope of the present invention. Advantages of the present invention described with reference to one exemplary embodiment also apply without being explicitly mentioned, where transferable, to other exemplary embodiments.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

What is claimed is:

1. A computer-implemented method for supplying a 3D model of a three-dimensional object represented by volume data, the method comprising:
supplying the volume data;
supplying an image synthesis algorithm, the image synthesis algorithm being configured for visualizing the three-dimensional object by mapping the volume data onto visualization pixels of a two-dimensional visualization image;
ascertaining a parameter set for actuating the image synthesis algorithm, the parameter set being suitable for generating, with the image synthesis algorithm, a set of a plurality of different visualization images based on the volume data;
generating the set of the plurality of different visualization images by mapping the volume data, with the image synthesis algorithm, using the parameter set;
calculating the 3D model based on the set of the plurality of different visualization images; and
supplying the 3D model.

2. The computer-implemented method as claimed in claim 1, wherein the calculating the 3D model utilizes photogrammetry.

3. The computer-implemented method as claimed in claim 1, wherein the parameter set comprises:
a first parameterization of mapping properties of the volume data; and
a second parameterization for generating a plurality of different views of the three-dimensional object with a same first parameterization to generate the plurality of different visualization images of the set.

4. The computer-implemented method as claimed in claim 3, wherein the first parameterization comprises a transfer function for at least one of mapping the volume data onto the two-dimensional visualization image or a segmenting of at least one of the three-dimensional object or a clipping mask.

5. The computer-implemented method as claimed in claim 1, wherein
the supplying of the volume data, the supplying of the image synthesis algorithm, the ascertaining of the parameter set, the generating of the set and the calculating of the 3D model are performed in a first computing device,
the supplying of the 3D model includes supplying the 3D model to a second computing device, the second computing device being different from the first computing device, and
the second computing device includes a portable end user device.

6. The computer-implemented method as claimed in claim 1, wherein the image synthesis algorithm implements a path tracing or ray casting method.

7. The computer-implemented method as claimed in claim 1, wherein
the volume data is generated by a medical imaging method and represents one or more organs of a patient,
the method includes supplying an item of patient information associated with the patient, and
the ascertaining ascertains the parameter set based on the patient information.

8. The computer-implemented method as claimed in claim 7, wherein the supplying of the 3D model comprises:
supplying the 3D model as further patient information for the patient.

9. The computer-implemented method as claimed in claim 7, further comprising:
determining one or more selected organs for presentation in the 3D model based on the patient information;
generating a segmenting mask for segmenting the one or more selected organs in the volume data; and wherein
the generating includes generating the plurality of different visualization images based on the segmenting mask.

10. The computer-implemented method as claimed in claim 7, wherein
the patient information includes an annotation based on the three-dimensional object, and
the generating includes inserting the annotation into the 3D model.

11. The computer-implemented method as claimed in claim 1, wherein the 3D model is supplied in a viewing application configured such that different views of the 3D model are selectable and viewable.

12. The computer-implemented method as claimed in claim 1, further comprising:
supplying a trained function configured to ascertain, based on the volume data, the parameter set for actuating an image synthesis algorithm; and wherein
the ascertaining ascertains the parameter set by applying the trained function.

13. An apparatus to supply a 3D model of a three-dimensional object represented by volume data, the apparatus comprising:
an interface configured to receive the volume data and to supply the 3D model; and
a computing device configured to
host an image synthesis algorithm, the image synthesis algorithm configured to visualize the three-dimensional object by mapping the volume data onto visualization pixels of a two-dimensional visualization image,
ascertain a parameter set for actuating the image synthesis algorithm, the parameter set being suitable for generating, with the image synthesis algorithm, a set of a plurality of different visualization images based on the volume data,
generate the set of the plurality of different visualization images by mapping the volume data, with the image synthesis algorithm, using the parameter set, and
calculate the 3D model based on the set of the plurality of different visualization images.

14. A non-transitory computer program product having a computer program, which is loadable into a storage device of an apparatus for supplying a 3D model, the computer program having program segments configured to carry out the computer-implemented method for supplying the 3D model as claimed in claim 1 when the program segments are executed by the apparatus.

15. A non-transitory computer-readable storage medium storing program segments that, when executed by an apparatus for supplying a 3D model, cause the apparatus to carry out the computer-implemented method for supplying the 3D model as claimed in claim 1.

16. The computer-implemented method of claim 8, wherein the further patient information is in the form of a medical report on diagnostic findings for the patient.

17. The computer-implemented method as claimed in claim 2, wherein
the supplying of the volume data, the supplying of the image synthesis algorithm, the ascertaining of the parameter set, the generating of the set and the calculating of the 3D model are performed in a first computing device,
the supplying of the 3D model includes supplying the 3D model to a second computing device, the second computing device being different from the first computing device, and
the second computing device includes a portable end user device.

18. The computer-implemented method as claimed in claim 3, wherein
the supplying of the volume data, the supplying of the image synthesis algorithm, the ascertaining of the parameter set, the generating of the set and the calculating of the 3D model are performed in a first computing device,
the supplying of the 3D model includes supplying the 3D model to a second computing device, the second computing device being different from the first computing device, and
the second computing device includes a portable end user device.

19. The computer-implemented method as claimed in claim 5, wherein
the volume data is generated by a medical imaging method and represents one or more organs of a patient,
the method includes supplying an item of patient information associated with the patient, and
the ascertaining ascertains the parameter set based on the patient information.

20. The computer-implemented method as claimed in claim 9, wherein
the patient information includes an annotation based on the three-dimensional object, and
the generating includes inserting the annotation into the 3D model.
